SEQUENCE LISTING (43 sequences)

SEQ ID NO: 1 - DNA, 736 bp, Homo sapiens
  CDS (57)...(596)
gaattcggcacgaggaggcgggcagcagctgcaggctgaccttgcagcttggcggaatggactggcctcacaacctgctgtttcttcttaccatttccatcttcctggggctgggccagcccaggagccccaaaagcaagaggaaggggcaagggcggcctgggcccctggcccctggccctcaccaggtgccactggacctggtgtcacggatgaaaccgtatgcccgcatggaggagtatgagaggaacatcgaggagatggtggcccagctgaggaacagctcagagctggcccagagaaagtgtgaggtcaacttgcagctgtggatgtccaacaagaggagcctgtctccctggggctacagcatcaaccacgaccccagccgtatccccgtggacctgccggaggcacggtgcctgtgtctgggctgtgtgaaccccttcaccatgcaggaggaccgcagcatggtgagcgtgccggtgttcagccaggttcctgtgcgccgccgcctctgcccgccaccgccccgcacagggccttgccgccagcgcgcagtcatggagaccatcgctgtgggctgcacctgcatcttctgaatcacctggcccagaagccaggccagcagcccgagaccatcctccttgcacctttgtgccaagaaaggcctatgaaaagtaaacactgacttttgaaagccagaaaaaaaaaaaaaaaaaaaaattcctgcggccgc

SEQ ID NO: 2 - protein, 180 aa, Homo sapiens
MDWPHNLLFLLTISIFLGLGQPRSPKSKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 3 - DNA, 397 bp, Homo sapiens
  misc_feature 10, 150, 267: n = A,T,C or G
aggcgggcanagctgcaggctgaccttgcagcttggcggaatggactggcctcacaacctgctgtttcttcttaccatttccatcttcctggggctgggcagccaggagccccaaaagcaagaggaaggggcaagggcggcctgggcccntggcctggcctcaccaggtgccactggacctggtgtcacggatgaaaccgtatgcccgcatggaggagtatgagaggaacatcgaggagatggtggcccagctgaggaacagctcanaagctggcccagagaaagtgtgaggtcaacttgcagctgtggatgtccaacaagaaggagcctgtctcccttggggctacaagcatcaaccaccgaccccagccgtatccccgtgggaccttgccgggac

SEQ ID NO: 4 - DNA, 18 bp, Artificial Sequence
  oligonucleotide primer
ttaccatttccatcttcc

SEQ ID NO: 5 - DNA, 18 bp, Artificial Sequence
  oligonucleotide primer
cccttcctcttgcttttg

SEQ ID NO: 6 - DNA, 29 bp, Artificial Sequence
  oligonucleotide primer
caaggatcccagcccaggagccccaaaag

SEQ ID NO: 7 - DNA, 30 bp, Artificial Sequence
  oligonucleotide primer
gacctcgagtcagaagatgcaggtgcagcc

SEQ ID NO: 8 - DNA, 30 bp, Artificial Sequence
  oligonucleotide primer
gtcgaattcatggactggcctcacaacctg

SEQ ID NO: 9 - DNA, 27 bp, Artificial Sequence
  oligonucleotide primer
gaaggatccgaagatgcaggtgcagcc

SEQ ID NO: 10 - protein, 10 aa, Artificial Sequence
  FLAG tag
DYKDDDDKGS

SEQ ID NO: 11 - DNA, 692 bp, Mus musculus
  CDS (50)...(589)
ggggttcctggcgggtggcagctgcgggcctgccgcctgacttggtgggatggactggccgcacagcctgctcttcctcctggccatctccatcttcctggcgccaagccacccccggaacaccaaaggcaaaagaaaagggcaagggaggcccagtcccttggcccctgggcctcatcaggtgccgctggacctggtgtctcgagtaaagccctacgctcgaatggaagagtatgagcggaaccttggggagatggtggcccagctgaggaacagctccgagccagccaagaagaaatgtgaagtcaatctacagctgtggttgtccaacaagaggagcctgtccccatggggctacagcatcaaccacgaccccagccgcatccctgcggacttgcccgaggcgcggtgcctatgtttgggttgcgtgaatcccttcaccatgcaggaggaccgtagcatggtgagcgtgccagtgttcagccaggtgccggtgcgccgccgcctctgtcctcaacctcctcgccctgggccctgccgccagcgtgtcgtcatggagaccatcgctgtgggttgcacctgcatcttctgagccaaccaccaacccggtggcctctgcaacaaccctccctccctgcacccactgtgaccctcaaggctgataaacagtaaacgctgttctttgtaaagga

SEQ ID NO: 12 - protein, 180 aa, Mus musculus
MDWPHSLLFLLAISIFLAPSHPRNTKGKRKGQGRPSPLAPGPHQVPLDLVSRVKPYARMEEYERNLGEMVAQLRNSSEPAKKKCEVNLQLWLSNKRSLSPWGYSINHDPSRIPADLPEARCLCLGCVNPFTMQEDRSMVSVPVFSQVPVRRRLCPQPPRPGPCRQRVVMETIAVGCTCIF

SEQ ID NO: 13 - DNA, 497 bp, Mus musculus
ggggttcctggcgggtggcagctgcgggcctgccgcctgacttggtgggatggactggccgcacagcctgctcttcctcctggccatctccatcttcctggcgccaagccacccccggaacaccaaaggcaaaagaaaagggcaagggaggcccagtcccttggcccctgggctcatcaggtgccgctggacctggtgtctcgagtaaagccctacgctcgaatggaagagtatgagcggaaccttggggagatggtggcccagctgaggaacagctccgagccagccaagaagaaatgtgaagtcaatctacagctgtggttgtccaacaagaggagcctgtccccatggggctacagcatcaaccacgaccccagccgcatccctgcggacttgcccgaggcgcggtgcctatgtttgggttgcgtgaatcccttcaccatgcaggaggaccgtagcatggtgagcgtgccagtgttcagccaggtgccggtgcg

SEQ ID NO: 14 - protein, 160 aa, Homo sapiens
QPRSPKSKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 15 - protein, 160 aa, Homo sapiens
QPRAPKSKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 16 - protein, 160 aa, Homo sapiens
QPRSPKAKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 17 - protein, 160 aa, Homo sapiens
QPRSPKSKRKGQGRPAPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 18 - protein, 160 aa, Homo sapiens
QPRSPKSKRKGQGRPGPLAPGPHQVPLDLVARMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 19 - protein, 160 aa, Homo sapiens
QPRSPKSKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRVVMETIAVGCTCIF

SEQ ID NO: 20 - protein, 160 aa, Homo sapiens
QPRSPKSKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRLVMETIAVGCTCIF

SEQ ID NO: 21 - protein, 160 aa, Homo sapiens
QPRSPKSKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRFVMETIAVGCTCIF

SEQ ID NO: 22 - protein, 160 aa, Homo sapiens
QPRSPKSKRKGQGRPGPLAPGPHQVPLDLVGRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 23 - protein, 160 aa, Homo sapiens
QPRSPKSKRKGQGRPSPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 24 - protein, 160 aa, Homo sapiens
QPRSPKVKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 25 - protein, 160 aa, Homo sapiens
QPRVPKSKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 26 - protein, 96 aa, Homo sapiens
CEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCI

SEQ ID NO: 27 - protein, 100 aa, Homo sapiens
PRSPKSKRKGQGRPGPLAPGPHQVPLDLVSRMKPYARMEEYERNIEEMVAQLRNSSELAQRKCEVNLQLWMSNKRSLSPWGYSINHDPSRIPVDLPEARC

SEQ ID NO: 28 - protein, 17 aa, Homo sapiens
PRSPKSKRKGQGRPGPL

SEQ ID NO: 29 - protein, 17 aa, Homo sapiens
RMKPYARMEEYERNIEE

SEQ ID NO: 30 - protein, 16 aa, Homo sapiens
NHDPSRIPVDLPEARC

SEQ ID NO: 31 - protein, 19 aa, Homo sapiens
PVRRRLCPPPPRTGPCRQR

SEQ ID NO: 32 - protein, 47 aa, Homo sapiens
PRSPKSKRKGQGRPGPLAPGPHQVPLDLVSRMKPYARMEEYERNIEE

SEQ ID NO: 33 - protein, 70 aa, Homo sapiens
RMKPYARMEEYERNIEEMVAQLRNSSELAQRKCEVNLQLWMSNKRSLSPWGYSINHDPSRIPVDLPEARC

SEQ ID NO: 34 - protein, 61 aa, Homo sapiens
NHDPSRIPVDLPEARCLCLGCVNPFTMQEDRSMVSVPVFSQVPVRRRLCPPPPRTGPCRQR

SEQ ID NO: 35 - protein, 73 aa, Homo sapiens
NHDPSRIPVDLPEARCLCLGCVNPFTMQEDRSMVSVPVFSQVPVRRRLCPPPPRTGPCRQRAVMETIAVGCTC

SEQ ID NO: 36 - protein, 158 aa, Homo sapiens
RSPKSKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 37 - protein, 154 aa, Homo sapiens
SKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 38 - protein, 151 aa, Homo sapiens
KGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 39 - protein, 160 aa, Mus musculus
HPRNTKGKRKGQGRPSPLAPGPHQVPLDLVSRVKPYARMEEYERNLGEMVAQLRNSSEPAKKKCEVNLQLWLSNKRSLSPWGYSINHDPSRIPADLPEARCLCLGCVNPFTMQEDRSMVSVPVFSQVPVRRRLCPQPPRPGPCRQRVVMETIAVGCTCIF

SEQ ID NO: 40 - protein, 158 aa, Mus musculus
RNTKGKRKGQGRPSPLAPGPHQVPLDLVSRVKPYARMEEYERNLGEMVAQLRNSSEPAKKKCEVNLQLWLSNKRSLSPWGYSINHDPSRIPADLPEARCLCLGCVNPFTMQEDRSMVSVPVFSQVPVRRRLCPQPPRPGPCRQRVVMETIAVGCTCIF

SEQ ID NO: 41 - protein, 153 aa, Homo sapiens
KRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 42 - protein, 128 aa, Homo sapiens
MKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 43 - protein, 157 aa, Homo sapiens
RSPKSKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCI

(19) United States
(12) Patent Application Publication
(10) Pub. No.: US 2008/0159979 A1
(43) Pub. Date: Jul. 3, 2008

(54) TREATMENT OF WOUNDS USING IL-17B

(76) Inventors: Emma E. Moore, Seattle, WA (US); Harald S. Haugen, Seattle, WA (US)

Correspondence Address: ZYMOGENETICS, INC.;INTELLECTUAL PROPERTY DEPARTMENT, 1201 EASTLAKE AVENUE EAST, SEATTLE, WA, 98102-3702, US

(21) Appl. No.: 11/557,315

(22) Filed: Nov. 7, 2006

Related U.S. Application Data
(63) Continuation-in-part of application No. 11/462,603, filed on Aug. 4, 2006.
(60) Provisional application No. 60/705,355, filed on Aug. 4, 2005.

(51) Int. Cl. A61K 38/20; A61K 39/395; A61P 17/06

(52) U.S. Cl. 424/85.2; 424/158.1

(57) ABSTRACT

IL-17B is known to stimulate the proliferation of chondrocytes, bone, and is highly expressed in nervous tissue, resulting in repair of diseased tissue. When IL-17B is absent a marked negative effect on wound healing is noted. The present invention comprises providing IL-17B, by topical, parental, or other administration means, in order to accelerate the wound healing process. The present invention further encompasses a pharmaceutical composition and formulations thereof that utilize IL-17B, either alone or in combination with other cytokines or growth factors known to aid wound healing. The invention also contemplates methods of treating wounds in patients using this pharmaceutical composition.

### CROSS-REFERENCE TO RELATED APPLICATIONS

This application is a continuation-in-part of U.S. application Ser. No. 11/462,603, filed Aug. 4, 2006, which claims the benefit of U.S. Provisional Application Ser. No. 60/705,355, filed Aug. 4, 2005, both of which are herein incorporated by reference.

### BACKGROUND OF THE INVENTION

### A. Wounds and Wound Healing

The human skin is composed of two distinct layers, the epidermis and dermis. Below these layers lies the subcutaneous tissue. The primary functions of these tissues are to provide protection, sensation, and thermoregulation to an animal. Secondarily, these layers protect the internal organs of the organism from shock or trauma by cushioning impacts from external forces and objects.

The outermost layer of skin, the epidermis, is approximately 0.04 mm thick, is avascular, is comprised of four cell types (keratinocytes, melanocytes, Langerhans cells, and Merkel cells), and is stratified into several epithelial cell layers (Leeson et al., (1985) Textbook of Histology, WB Saunders Co., Philadelphia). The inner-most epithelial layer of the epidermis is the basement membrane, which is in direct contact with, and anchors the epidermis to, the dermis. All epithelial cell division occurring in skin takes place at the basement membrane. After cell division, the epithelial cells migrate towards the outer surface of the epidermis. During this migration, the cells undergo a process known as keratinization, whereby nuclei are lost and the cells are transformed into tough, flat, resistant non-living cells. Migration is completed when the cells reach the outermost epidermal structure, the stratum corneum, a dry, waterproof squamous cell layer that helps to prevent desiccation of the underlying tissue. This layer of dead epithelial cells is continuously being sloughed off and replaced by keratinized cells moving to the surface from the basement membrane. Because the epidermal epithelium is avascular, the basement membrane is dependent upon the dermis for its nutrient supply.

The dermis is a highly vascularized tissue layer supplying nutrients to the epidermis. In addition, the dermis contains nerve endings, lymphatics, collagen protein, and connective tissue. The dermis is approximately 0.5 mm thick and is composed predominantly of fibroblasts and macrophages. These cell types are largely responsible for the production and maintenance of collagen, the protein found in all animal connective tissue, including the skin. Collagen is primarily responsible for the skin's resilient, elastic nature. The subcutaneous tissue, found beneath the collagen-rich dermis, provides for skin mobility, insulation, calorie storage, and blood to the tissues above it.

Whenever there is an injury to the skin and/or the underlying soft tissue, a process to repair the resultant wound is immediately initiated in healthy organisms. In humans, this process does not lead to total regeneration of the injured outer integument unless the injury is confined to the epidermis and the basement membrane is left intact (Wokalek, H., (1988) CRC Critical Reviews in Biocompatibility, vol. 4, issue 3: 209-46). Therefore, when a wound is characterized by more extensive tissue damage, the injured, destroyed, or lost tissue will not be reconstituted with like tissue, but will instead be replaced by scar tissue. Wounds characterized by tissue disruption penetrating completely through both the epidermis and dermis are known as full thickness wounds, while those which only extend through the epidermis but do not completely pass through the dermis are called partial thickness wounds.

Wound healing is the process through which the repair of damaged tissue(s) is accomplished. Wounds in which there is little or no tissue loss are said to heal by first or primary intention, while deep wounds suffering tissue loss heal by second or secondary intention. The wound healing process is comprised of three different stages, referred to as inflammation, granulation tissue formation, and matrix formation and remodeling (Ten Dijke et al., (1989) Biotechnology, vol. 7: 793-98).

The inflammatory response to soft tissue injury is initiated immediately upon infliction of the wound as tissue edges are separated and blood spills into the wound, activating the clotting cascade, leading to hemostasis. Initially there is a short phase of vasodilation in tissues surrounding the wound site followed by vasoconstriction. Platelets present in the wound, which aggregate to form the clot, also release a number of vasoactive compounds, chemoattractants, and growth factors (Goslen, J. B., (1988) J. Dermatol. Surg. Onco., vol 9: 959-72). The clot itself is critical for eventual wound repair, as the provisional fibronectin matrix is used by fibroblasts and epithelial cells for ingress into the wound. Additionally, capillary permeability peripheral to the wound is increased, and because of the reduced blood flow, polymorphonuclear leukocytes (PMNs) adhere to the capillary walls and migrate into the wound, as do monocytes (Eckersley et al., (1988) British Medical Bulletin, vol. 44, No. 2: 423-36).

PMNS, such as neutrophils, are the predominant cell type found in the wound initially. PMNs and macrophages begin the process of cleaning the wound. This cleansing process is accomplished mostly through the phagocytosis of devitalized tissue and other debris. By days 3-5 post-injury, PMNs have largely been replaced by macrophages, which continue to remove dead and foreign material. In 1972, Simpson and Ross (J. Clin. Invest., vol 51: 2009-23) showed that an almost total absence of PMNs in the wound site did not inhibit wound healing. However, the role of macrophages in wound repair may be critical (Diegelmann et al., (1981) Plast. Reconstr. Surg., vol. 68: 107-113). In experimental monocytopenic wounds, granulation tissue formation, fibroplasia, and collagen disposition are markedly impaired and healing is delayed (Leibovich et al., (1975) Am. J. Path., vol 78: 71-100; Mustoe et al., (1989) Am. J. Surg., vol 158: 345-50; Pierce et al., (1989) Proc. Nat. Acad. Sci. USA, vol. 86: 2229-33).

When found in wounds, macrophages are known to release a variety of biologically active substances that serve as chemoattractants for both monocytes and fibroblasts, such as transforming growth factor-beta (TGF-β) and platelet-derived growth factor (PDGF) (Rappollee et al., (1988) Science, vol. 241: 708-12; Pierce et al., supra; Pierce et al., (1989) J. Cell Biol., vol. 109: 429-40). See Obberghen-Schilling et al., (1988) J. Biol. Chem., vol. 263: 7741-46; Paulsson et al., (1987) Nature, vol. 328: 715-17; and Coffey et al., (1987) Nature, vol. 328: 817-20). Activated macrophages digest devitalized collagen and the fibrin clot. Dissolution of the clot allows the formation of granulation tissue in the wound site, the second wound-healing phase.

Granulation tissue formation begins three to four days after the injury is inflicted and continues in the open wound until re-epithelialization has occurred. This stage is marked by the proliferation of fibroblasts and their migration into the wound site where they then produce an extracellular matrix, known as ground substance, comprised of collagen, fibronectin, and hyaluronic acid to replace the digested clot. This extracellular matrix serves as a scaffold upon which endothelial cells, fibroblasts, and macrophages are able to move. It is also utilized by myofibroblasts to promote wound closure by the process of wound contraction in full thickness wounds which heal by secondary intent.

Myofibroblasts are derived through the differentiation of resident fibroblasts shortly after a full thickness wound is inflicted. These myofibroblasts align radially using the newly deposited extracellular matrix and in an association with matrix, called the fibronexus, contract and promote more rapid wound closure (Singer et al., (1984) J. Cell Biol., vol. 98: 2091-2106).

In addition to wound closure, reepithelialization also occurs during this stage of wound healing. Epithelial cells proliferate at the wound edges and migrate across the ground substance. Epithelial cells can move only over viable, vascular tissue. Migration is halted by contact inhibition among epithelial cells, which at this point divide and differentiate to reconstitute the epithelium (Hunt et al., (1979) Fundamentals of wound management, Appleton-Century-Crofts).

As granulation tissue formation proceeds, angiogenesis, the formation of new blood vessels produced by endothelial cell division and migration, also occurs as the result of hypoxic conditions in the wound. Knighton et al. ((1983) Science, vol. 221: 1283-85) showed that macrophages, under hypoxic conditions, stimulate angiogenesis. The resultant increased vascularization increases blood flow and oxygenization in the wound. Eventually, as wound healing progresses into the matrix formation and remodeling phase, much of this newly formed vasculature regresses to leave a relatively avascular scar.

1'Collagen and matrix remodeling begin when granulation tissue formation begins and continues long after the wound has been covered by new epithelium and can continue for more than a year. This final stage of wound healing is characterized by devascularization and the replacement of granulation tissue and cells with a matrix comprised predominantly of type I collagen. This new relatively acellular, avascular tissue represents the scar. Scar formation primarily serves to restore tensile strength to the wounded tissue. However, the scar will not possess more than about 80% of the tensile strength that the tissue had prior to being injured.

### B. Interleukins and the IL-17 Family

The Interleukins (ILs) are a polypeptide family playing a major role in the body's immune response. The IL-17 family is a subgroup of five interleukins that show 50-70% sequence homology to the first discovered member, IL-17, now named IL-17A. All share conserved cysteines that have been shown (at least for IL-17F) to form a classic cysteine knot structural motif found in other growth factors such as bone morphogenetic proteins (BMPs), transforming growth factor beta (TGF-β), nerve growth factors (NGF), and platelet-derived growth factor BB (PDGF-BB) (Hymowitz et al., (2001) EMBO J. 20(19):5332-41). IL-17A and IL17-F, as is typical for interleukins, are primarily expressed in T-cells in response to antigenic and mitogenic stimulation. In contrast, IL-17B, IL-17C, IL-17D, and IL-17E are expressed in a wide assortment of tissues (Moseley et al., (2003) Cytokine & Growth Factor Rev. 14: 155-174). Similar to many growth factors, members of the IL-17 family of ligands are expressed as tightly associated dimers (IL-17B; Shi et al. (2000) J. Biol. Chem. 275 (25): 19167-76) or disulfide-bonded homodimers (IL-17D; Stames et al. J. Immunol.).

IL-17B (also known as zcyto7, CX1, and NERF) is strongly expressed in spinal cord tissue, specifically neurons and dorsal root ganglia, and weakly expressed in the trachea. Administration of the protein in vitro stimulates the proliferation of chondrocytes and osteoblasts. The gene is located on chromosome 5q32. It has been described extensively in U.S. Pat. Nos. 6,528,621; 6,500,928, and 6,630,571, the descriptions of which are hereby incorporated by reference. Other investigators have reported expression in adult pancreas, small intestine, and stomach and that it can induce the expression of tumor necrosis factor alpha (TNF-α) and interleukin 1 beta (IL-1β) from a monocytic cell line (Li et al., (2000) PNAS 97:773-8).

### C. Current Methods to Promote Wound Healing

Excluding infection or other complications, the normal wound healing process often results in the complete restoration of tissue function. Classically, the medical profession has been limited in what it can do to promote wound healing. In the past, such activities have been limited to the cleansing and debridement of the initial wound, suturing the wound or grafting skin if appropriate, dressing the wound to prevent desiccation and infection, and applying antibiotics, either locally or systemically, to reduce the risk of infection. Such treatment has been designed to provide optimal conditions for the natural healing process.

It has been noted that a number of cytokines and/or growth factors may accelerate the wound healing process, in both acute and chronic wounds, in animal models. These derived factors include Platelet-Derived Growth Factor (PDGF), Fibroblast Growth Factor (FGF), Epidermal Growth Factor (EGF), Hematopoietic Colony Stimulating Factor (CSF), Granulocyte Macrophage Colony Stimulating Factor (GM-CSF) and Transforming Growth Factors-α and -β (TGF-α and TGF-β). Additionally, other growth factors, including interleukins (ILs) other than IL-17B, insulin, Insulin-like Growth Factors I and II (IGF-I and IGF-II, respectively), Interferons (IFNs), KGF (Keratinocyte Growth Factor), Macrophage Colony Stimulating Factor (M-CSF), Platelet-Derived Endothelial Cell Growth Factor (PD-ECGF), and Stem Cell Factor (SCF), may promote the activation, proliferation, and/or stimulation of cell types involved in the wound healing process.

Because each of these growth factors mentioned above may be capable of acting as a mitogen, inhibitor, or chemoattractant for the cell types heavily involved in the wound healing process, i.e. monocyte/macrophage, neutrophil, fibroblast, and endothelial and epithelial cells, they have been studied extensively in animal wound healing models. The most studied growth factor in relation to wound healing, EGF, has been found to accelerate the healing of surface wounds and bums when repeatedly applied to the wound site. PDGF and TGF-β increase the healing rate of incisional wounds when administered one time to the incision site shortly after the wound is made. However, no work describing the use of other factors, such as members of the IL-17 family, can be found in the literature.

Thus, the object of the present invention is to provide a method for accelerating the wound healing process. Relating to wounds that will heal normally, the described method will accelerate this process. Concerning wounds that typically resist healing, this method will enable healing of these wounds as well. This method should reduce the time required for injury repair, and as such will lessen the time those burdened with injury will have to endure as their wounds heal.

### SUMMARY OF THE INVENTION

The present invention provides for a method of promoting accelerated wound healing in an injured patient by administering a therapeutically effective amount of IL-17B to the patient at the wounded area. This can be accomplished by incorporating the therapeutic agent into various materials, including: collagen based creams, films, microcapsules, or powders; hyaluronic acid or other glycosaminoglycan-derived preparations; creams, foams, suture material; and wound dressings. Alternatively, the therapeutic agent can be incorporated into a pharmaceutically acceptable solution designed for topical administration. Further, the therapeutic agent can be formulated for parenteral administration.

The methods of the present invention are effective in accelerating wound healing in incisional, compression, thermal, acute, chronic, infected, and sterile injuries.

Additionally, IL-17B can also be incorporated into an admixture containing at least one of the following proteins: GM-CSF, CSF, EGF, FGF, G-CSF, IGF-I, IGF-II, insulin, an Interferon, an Interleukin, KGF, M-CSF, PD-ECGF, PDGF, SCF, TGF-α, and TGF-β. These admixtures are also effective in promoting accelerated wound healing in injured patients.

### BRIEF DESCRIPTION OF THE FIGURES

FIG. 1 is a graphical representation of the occurrence of heightened redness surrounding the wounds for wild-type and IL-17B (zcyto7) knockout mice at two time points.

FIG. 2 indicates the fold overexpression of various cytokines/growth factors at the RNA level in the knockout mice as compared to wild-type.

FIG. 3 indicates the underexpression of various genes associated with normal fully differentiated epidermis at the RNA level in the knockout mice as compared to wild-type.

### DETAILED DESCRIPTION

Because the typical wound is localized, cell types needed to effect wound repair must be concentrated in and around the injured area. Thus it is preferable that the factors necessary to promote the wound healing activity of these cell types be present in the afflicted area. Topical delivery of the polypeptide(s) is the most efficient way to achieve these goals.

The instant invention is based upon the discovery that Il-17B can accelerate the wound healing process for all wound types, particularly when administered topically, i.e. to the surface of the wound site. So delivered, all wound types, mechanical or thermal, acute or chronic, infected or sterile, undergo healing more rapidly than similar wounds left to heal naturally or which are treated with currently available methods. However, as mentioned previously, parenteral administration of polypeptides having a role in the wound healing process is also envisioned by the present invention.

In accordance with the present invention, the term “injury” shall be defined as a wound which extends from the surface of a patient's skin into the underlying tissue, and in fact the injury may pass completely through the patient, leaving both entrance and exit wounds. “Patient” refers to a mammal which has suffered an injury as defined above. “Therapeutic agent” means a compound that produces a therapeutically desirable result, such as accelerated wound healing. In the present invention, the therapeutic agent is IL-17B (zcyto7). Additionally, the term “therapeutic agent” refers to a combination of IL-17B combined with at least one of the following compounds: a CSF, EGF, FGF, IGF-I, IGF-II, insulin, an Interferon, an Interleukin, KGF, M-CSF, PD-ECGF, PDGF, SCF, TGF-α, and TGF-β. Here, “accelerated wound healing” is defined as the process of wound healing which, as the result of the administration of a therapeutic agent in accordance with the present invention, occurs more rapidly than in a wound not receiving treatment with the therapeutic agent.

CSFs are hormone-like glycoproteins which regulate hematopoiesis and are required for the clonal growth and maturation of normal hematopoietic precursor cells found in the bone marrow. These factors are produced by a number of tissues. Four CSFs isolated from human sources have been identified: granulocyte colony stimulating factor (G-CSF) [Welte et al., (1985) Proc. Nat. Acad. Sci. USA, vol. 82: 1526-30]; granulocyte-macrophage colony stimulating factor (GM-CSF) [Cantrell et al., (1985) Proc. Nat. Acad. Sci. USA, vol. 82: 6250-54]; macrophage colony stimulating factor (M-CSF); and multi-colony stimulating factor (multi-CSF, also referred to as Interleukin-3 [Nicola et al., (1984) Proc. Nat. Acad. Sci. USA, vol. 81: 3765-69], each accounting for the differentiation of particular immature progenitor cell types into mature cells. In addition, these factors are required for the maintenance of the mature cell types as well. In vitro, withdrawal of the appropriate CSF from culture leads to rapid degeneration of terminally differentiated hematopoietic cells dependent upon that CSF. Two particular CSFs that can be combined with IL-17B are G-CSF and GM-CSF.

EGF is a polypeptide growth factor (the mature, processed form is 53 amino acids in length (Gray et al., (1983) Nature, vol. 303: 722-25)). In humans, this protein inhibits gastric acid secretion while murine EGF is known to be mitogenic for a number of cell types, including endothelial, epithelial, and fibroblastic cells (Nakagawa et al., (1985) Differentiation, vol. 29: 284-88).

FGF comprises a family of single chain proteins 14-18 kD in size which tightly bind the potent anticoagulant heparin. Two FGF types, acidic and basic, have been reported. The 146 amino acid basic form (bFGF) is more stable and ten times more potent in stimulating mesodermal cells, such as fibroblasts, endothelial cells, and keratinocytes, than acidic FGF (aFGF). See Esch et al., (1985) Proc. Nat. Acad. Sci. USA, vol. 85: 6507-11).

Insulin is a protein hormone secreted by the cells of the pancreatic islets. It is secreted in response to elevated blood levels of glucose, amino acids, fatty acids, and ketone bodies, promoting their efficient storage and use as cellular fuel by modulating the transport of metabolites and ions across cell membranes and by regulating various intracellular biosynthetic pathways. Insulin promotes the entry of glucose, fatty acids, and amino acids into cells. Additionally, it promotes glycogen, protein, and lipid synthesis while inhibiting glucogenesis, glycogen degradation, protein catabolism, and lipolysis. Insulin consists of α and β subunits linked by two disulfide bridges.

IGF-I and IGF-II are members of a growth hormone-dependent family which mediate the effects of growth hormones. These proteins are known to be important in the regulation of skeletal growth. Both molecules have close structural homology to insulin and possess similar biological activities. IGF-I shares a 43% amino acid sequence homology with proinsulin, while IGF-II shares 60% homology with IGF-I. The IGFs are somewhat unique as compared to the other proteins described herein, in that there is essentially no detectable free IGF species present in the blood plasma of mammals. Instead, the IGFs are bound to specific carrier plasma proteins of higher molecular weight (Ooi et al., (1988) J. Endocr., vol. 118:7-18). Both IGF species stimulate DNA, RNA, and protein synthesis and are involved in the proliferation, differentiation, and chemotaxis of some cell types. Local administration of IGF-I is known to stimulate the regeneration of peripheral nerves. In addition, IGF-I and PDGF, when administered topically to wounds in pigs, synergize to promote more effective healing than when either factor is administered alone (Skoffner et al., (1988) Acta. Paediatr. Scand. (Suppl), vol. 347:110-12).

Interferons were first identified as proteins that render cells resistant to infection from a wide range of viruses. Three Interferon types have been identified, α-IFN, β-IFN, and γ-IFN, which are produced by activated T and NK (natural killer) cells. α-IFN is comprised of a family of 15 or so closely related proteins while β-IFN and γ-IFN exist as single species. In addition, a synthetic consensus α-IFN, designed to incorporate regions of commonality among all known α-IFN subtypes, is disclosed in U.S. Pat. No. 4,897,471, hereby incorporated by reference. All IFNs are growth inhibitory molecules playing an important role in the lymphokine cascade. Each exerts a wide range of regulatory actions in normal cells, cancer cells, and host immune defense cells. γ-IFN's activities include macrophage activation for enhanced phagocytosis and tumor killing capacity. At present, these proteins are mainly used in cancer therapy (Balkhill et al., (1987) Lancet, pg: 317-18).

KGF is an epithelial cell specific mitogen secreted by normal stromal fibroblasts. In vitro, it has been demonstrated to be as potent as EGF in stimulating the proliferation of human keratinocytes (Marchese et al., (1990) J. Cell Physiol., vol. 144, No. 2: 326-32).

M-CSF, also known as CSF-1, is a homodimeric colony stimulating factor which acts solely on macrophage progenitors. This macrophage lineage specific protein is produced constitutively in vitro by fibroblasts and stromal cell lines. In vivo, unlike other CSFs, M-CSF appears early in embryogenesis, suggesting a potential developmental role for this polypeptide (DeLamarter, J., (1988) Biochemical Pharmacology, vol. 37, No. 16: 3057-62).

PD-ECGF is a platelet derived endothelial cell mitogen having a molecular weight of approximately 45 kD. In contrast to the FGF family of endothelial cell mitogens, PD-ECGF does not bind heparin nor does it induce fibroblast proliferation. However, PD-ECGF does stimulate endothelial cell growth and chemotaxis in vitro and angiogenesis in vivo (Ishikawa et al., (1989) Nature, vol. 338: 557-61).

PDGF is a potent stimulator of mesenchymal cell types, like fibroblasts and smooth muscle cells, but it does not stimulate the growth of epithelial or endothelial cells (Ross et al., (1986) Cell, vol. 45: 155-69). At low concentrations, PDGF acts as a chemoattractant for fibroblasts, and also as a chemoattractant and activating signal for monocytes and neutrophils (Deuel et al., (1982) J. Clin. Invest., vol. 69: 1046-49).

SCF is a novel cellular growth factor that stimulates the growth of early hematopoietic progenitor cells, neural stem cells, and primordial germ stem cells (PCT/US90/05548, filed Sep. 28, 1990). SCF exhibits potent synergistic activities in conjunction with colony stimulating factors, resulting in increased numbers of colonies and colonies of greater size (Martin et al., (1990) Cell, vol. 63: 203-11). Thus, administration of SCF to mammals in pharmacologic doses, alone or in combination with other colony stimulating factors or other hematopoietic growth factors, may lead to the improvement of damaged cells in a number of divergent organ systems.

TGF-α and TGF-β act synergistically to induce anchorage independent growth in certain cancer cell lines. TGF-β is comprised of a class of disulfide linked homodimeric proteins, each chain being composed of 112 amino acids (Spom et al., (1987) J. Cell Biol., vol. 105: 1039-45). This dimeric protein produces many biological effects, such as mitogenesis, growth inhibition, and differentiation induction depending upon the assay used. TGF-β1 is the most studied TGF-β species in relation to wound healing (Ten Dijke, supra). As a class, TGF-β is a potent monocyte and fibroblast chemoattractant.

“Topical administration” shall be defined as the delivery of the therapeutic agent to the surface of the wound and adjacent epithelium. “Parenteral administration” is the systemic delivery of the therapeutic agent via injection to the patient. A “therapeutically effective amount” of a therapeutic agent within the meaning of the present invention will be determined by a patient's attending physician or veterinarian. Such amounts are readily ascertained by one of ordinary skill in the art and will enable accelerated wound healing when administered in accordance with the present invention. Factors which influence what a therapeutically effective amount will be include, the specific activity of the therapeutic agent being used, the wound type (mechanical or thermal, full or partial thickness, etc.), the size of the wound, the wound's depth (if full thickness), the absence or presence of infection, time elapsed since the injury's infliction, and the age, physical condition, existence of other disease states, and nutritional status of the patient. Additionally, other medication the patient may be receiving will effect the determination of the therapeutically effective amount of the therapeutic agent to administer. “Pharmaceutically acceptable” means that the components, in addition to the therapeutic agent, comprising the formulation are suitable for administration to the patient being treated in accordance with the present invention.

In accordance with the present invention, “wound dressings” are any of a variety of materials utilized for covering and protecting a wound. Examples include occlusive dressings, adhesive dressings, antiseptic dressings, and protective dressings. In pharmaceutical preparations, a “cream” is a semisolid emulsion of the oil-in-water or water-in-oil type suitable for topical administration. In accordance with the present invention, creams and foams used will also be suitable for use with the therapeutic agents herein described.

IL-17B, when administered as taught by the present invention in a therapeutically effective amount, significantly accelerates the wound healing process in all wound types. In natural wound systems, extracellular growth factors such as IL-17B may be present in rate limiting quantities. Thus, parenteral and/or topical administration of such factors may promote accelerated wound healing.

In vitro IL-17B, is known to stimulate the proliferation of chondrocytes and osteoblasts. It may also induce the expression of other cytokines such as TGF-α and IL-1β. In vivo, administration of exogenous IL-17B is believed to enhance an organism's ability to respond to injury.

Any analogs of IL-17B possessing comparable or enhanced in vivo biological activity can be used in accordance with the methods of the present invention. IL-17B is preferably produced by recombinant methods which allows for alteration of the molecule to produce an analog. Such analogs may be generated by the deletion, insertion, or substitution of amino acids in the primary structure of the naturally occurring proteins, or by chemical modification, such as by pegylation, of the protein. For example, to enable expression of these polypeptides in procaryotic host microorganisms, an initial methionine codon is required for translation initiation. Other analogs may have greater in vitro and/or in vivo biological activity, exhibit greater pH or temperature stability, maintain biological activity over a broader range of environmental conditions, or may have longer half-lives or clearance times in vivo.

To manufacture sufficient quantities of IL-17B for commercial pharmaceutical application, these proteins are generally produced as the products of recombinant host cell expression. It is known that biologically active forms of IL-17B can be recovered in large quantities from procaryotic hosts such as E. coli when such hosts, transformed with appropriate expression vectors encoding these polypeptides, are grown under conditions allowing expression of the exogenous gene. It is therefore preferred to utilize IL-17B produced in this manner.

The recombinant IL-17B is formulated into a pharmaceutical formulation suitable for patient administration. As will be appreciated by those skilled in the art, such formulations may include pharmaceutically acceptable adjuvants and diluents. When administered systemically, a therapeutically effective amount of the therapeutic agent is delivered by the parenteral route, i.e. by subcutaneous, intravenous, intramuscular, or intraperitoneal injection. Wound treatment by parenteral injection may involve either single, multiple, or continuous administration of the therapeutic agent, depending upon various factors, including the injury type, severity, and location.

The amount of topical IL-17B to be administered can be determined by one of ordinary skill, but would be expected to range from about 0.05 to about 100 μg/cm² of IL-17B with the expected most effect range to be about 10 to about 75 μg/cm². In a preferred embodiment, the dosage is 50 μg/cm². Other modes of administration, such as parenteral, i.e., intramuscular or subcutaneous, would expected to be lower and based on pg per kg of patient body weight.

In a preferred embodiment of the present invention, recombinant IL-17B should be topically administered to the wound site to promote accelerated wound healing in the patient. This topical administration can be as a single dose or as repeated doses given at multiple designated intervals. It will readily be appreciated by those skilled in the art that the preferred dosage regimen will vary with the type and severity of the injury being treated. For example, surgical incisional wounds cause little damage to surrounding tissues, as little energy is transmitted to the tissues from the object inflicting the injury. It has been found that a single topical administration of the therapeutic agent results in significantly more rapid healing than in identical wounds which go untreated. Where the wound is infected and chronically granulating, repeated daily application of the therapeutic agent has been found to produce more rapid wound healing than in similar wounds receiving no treatment. IL-17B will be particularly useful in patients already suffering from diabetes, as glucose inbalance does make the patient more succeptible to skin injury.

While it is possible to administer the therapeutic agent as a pure or substantially pure compound, i.e. not incorporated into any pharmaceutical formulation, it is preferable instead to present the therapeutic agent in a pharmaceutical formulation or composition. Such formulations comprise a therapeutically effective amount of the therapeutic agent with one or more pharmaceutically acceptable carriers and/or adjuvants. The carriers employed must be compatible with the other ingredients in the formulation. Preferably, the formulation will not include oxidizing or reducing agents or other substances known to be incompatible with the described polypeptides. All formulation methods include the step of bringing the biologically active ingredient into association with the carrier(s) and/or adjuvant(s). In general, the therapeutic agent of the instant invention will be formulated by bringing the agent into association with liquid carriers, finely divided solid carriers, or both.

Formulations suitable for topical administration in accordance with the present invention comprise therapeutically effective amounts of the therapeutic agent with one or more pharmaceutically acceptable carriers and/or adjuvants. An aqueous or collagen-based carrier vehicle is preferred for topical administration of the therapeutic agents described by the present invention. When the formulation is to be administered but one time, a collagen-based carrier vehicle is preferred. An example of such a vehicle is Zyderm.™. (Collagen Corp., Palo Alto, Calif.). If the wound being treated requires multiple applications of the therapeutic agent at designated intervals, it is preferred to utilize a pharmaceutically acceptable aqueous vehicle for delivery. However, it is also possible to incorporate the therapeutic agent into a variety of materials routinely used in the treatment of wounds. Such materials include hyaluronic acid or other glycosaminoglycan-derived preparations, sutures, and wound dressings.

When the therapeutic agent used in accordance with the present invention is comprised of more than one protein, the resultant admixture is commonly administered in the same fashion as formulations comprising only one polypeptide as the therapeutic agent.

One specific use of IL-17B in the wound healing area is the use in treating mucositis associated with chemotherapy. Mucositis occurs when cancer treatments break down the rapidly divided epithelial cells lining the GI tract, particularly in the oral cavity, leaving the mucosal tissue open to ulceration and infection. Mucositis can occur anywhere along the digestive tract from the mouth to the anus. Oral mucositis is probably the most common debilitating complication of cancer surgery, chemotherapy, and radiation. It occurs in 20-40% of patients treated with chemotherapy alone and up to 50% of patients receiving combination radiation and chemotherapy, especially those with head and neck cancer. Drugs such as doxorubicin, paclitaxel, and capecitabine are commonly used in breast cancer and frequently associated with oral and GI mucositis. The consequences of mucositis can be mild requiring little intervention to severe (hypovolemia, electrolyte abnormalities, and malnutrition) that may result in fatal complications. Based on the wound healing effects on IL-B knockout mice described below in the examples, administration of IL-17B to patients undergoing chemotherapy or radiation therapy will prophylactically prevent such a syndrome or alternatively, speed healing from those suffering from mucositis due to cancer treatments.

A further specific use IL-17B is the treatment and/or prevention of pressure ulcers. A pressure ulcer is an area of skin that breaks down when a patient stays in one position for too long without shifting your weight. This is common with those that use a wheelchair or are bedridden, even for a short period of time (for example, after surgery or an injury). The constant pressure against the skin reduces the blood supply to that area, and the affected tissue dies. A pressure ulcer starts as reddened skin but gets progressively worse, forming a blister, then an open sore, and finally a crater. The most common places for pressure ulcers are over bony prominences (bones close to the skin) like the elbow, heels, hips, ankles, shoulders, back, and the back of the head. Based on the wound healing effects on IL-B knockout mice described below in the examples, administration of IL-17B to patients at risk for pressure ulcers will prophylactically prevent such sores or alternatively, speed healing from those already suffering from pressure ulcers.

An additional use for IL-17B is in the treatment of Inflammatory Bowel Disease (IBD). IBD refers to two chronic diseases that cause inflammation of the intestines: ulcerative colitis and Crohn's disease. Although the diseases have some features in common, there are some important differences. Ulcerative colitis is an inflammatory disease of the large intestine, also called the colon. In ulcerative colitis, the inner lining—or mucosa—of the intestine becomes inflamed and develops ulcers. Ulcerative colitis is often the most severe in the rectal area, which can cause frequent diarrhea. Mucus and blood often appear in the stool if the lining of the colon is damaged. Crohn's disease differs from ulcerative colitis in the areas of the bowel it involves—it most commonly affects the last part of the small intestine, the terminal ileum, and parts of the large intestine. However, Crohn's disease isn't limited to these areas and can attack any part of the digestive tract. Crohn's disease causes inflammation that extends much deeper into the layers of the intestinal wall than ulcerative colitis does. Crohn's disease generally tends to involve the entire bowel wall, whereas ulcerative colitis affects only the lining of the bowel. Based on the wound healing effects on IL-B knockout mice described below in the examples, administration of IL-17B to patients with ulcerative colitis or Crohn's Disease will speed healing from the damage present in the intestinal tracts of these patients.

IL-17B treatment may also be useful in the treatment of diseases where epithelial repair and/or proliferation have been dysregulated. One example of such a disease is psoriasis. Psoriasis is a noncontagious, lifelong skin disease. According to the National Institutes of Health, as many as 7.5 million Americans have psoriasis. The most common form, plaque psoriasis, appears as raised, red patches or lesions covered with a silvery white buildup of dead skin cells, called scale. The production of the scale is throught to be a result of dysregulation of the usual wound healing responses in the epidermis. It is believed that psoriasis develops when the immune system tells the body to over-react and accelerate the growth of skin cells. Normally, skin cells mature and are shed from the skin's surface every 28 to 30 days. When psoriasis develops, the skin cells mature in 3 to 6 days and move to the skin surface. Instead of being shed, the skin cells pile up, causing the visible lesions. In particular, antagonists to IL-17B may be useful for treatment of this dysregulation of the skin cell growth cycle.

The invention is further illustrated by the following non-limiting examples.

### EXAMPLE 1

Wild type or IL-B (zcyto7) homozygous knockout mice were anesthetized with isoflourane and the dorsum shaved and depilated. After 24 hrs mice were anesthetized with isoflourane, and the dorsum cleaned with Povidone-Iodine and Isopropyl alcohol pads. Animals received either one or two full thickness wounds of 0.5 cm² or 1 cm²; these were induced on either flank by the surgical removal of a piece of full thickness dorsal skin. The wound area was then bandaged with a Johnson & Johnson Bioocclusive dressing and these dressings were removed at three days. Animals were examined daily and the size and physical appearance of the wounds assessed. At various time points a 1 cm² area of skin surrounding the 0.5 cm² wound was surgically removed and these samples were processed for histological evaluation by formalin fixation or flash frozen in liquid nitrogen for RNA isolation. At various time points, final size and appearance observations were made. The animals were then euthanized and skin surrounding both wounds was collected for histological evaluation and RNA isolation as described in Example 2.

For histological evaluation, samples were paraffin embedded and stained with hematoxylin and counterstained with eosin by standard techniques. These sections were then scored by light microscopy. In one study, histological evaluation of IL-17B (zcyto7) knockout mice revealed decreased granulation tissue and reduced epithelial migration into the area of the wound bed, consistent with a reduced healing response in knockout animals.

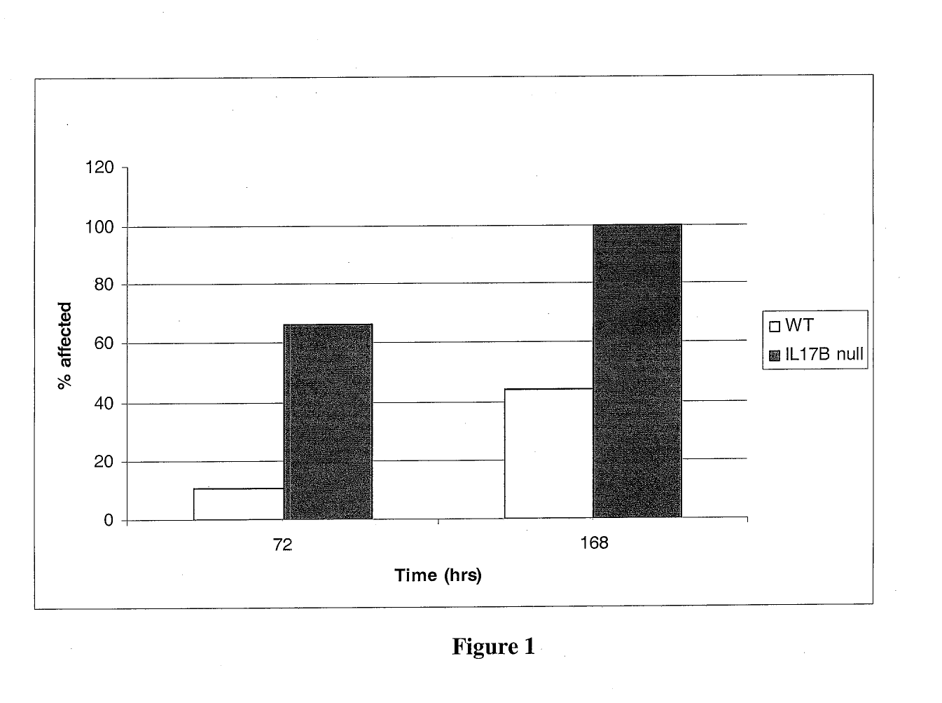

Visual assessment of wound beds from two separate wound-healing experiments also indicated that IL-17B (zcyto7) knockout mice exhibited increased swelling and redness of the tissue surrounding the wound bed both at early and late time points post wounding, suggesting that inflammatory responses were elevated and sustained in these animals. FIG. 1 graphically represents the observational results of one of these experiments. As indicated in the figure, a much higher percentage of knock-out mice exhibited unusual redness around the wound at both time points when compared to wild-type controls.

### EXAMPLE 2

The observational experiments of Example 1 were supported by RNA-based expression measurements. Using a multiplex approach, the expression of 293 genes in normal and wounded tissue from wild type and knockout mice were examined. Multiplex gene expression assays of murine skin tissue samples were performed essentially as described by Yang et al. (Yang et al., “BADGE, BeadsArray for the Detection of Gene Expression, a High-Throughput Diagnostic Bioassay”, GenomeResearch, 11:1888-1898 (2001)). Total RNA was prepared using a standard phenol:chloroform extraction protocol for tissues and converted to antisense RNA (aRNA) using Ambion MessageAmp aRNA Amplification kits (Ambion, Inc. Austin, Tex.), incorporating biotinylated UTP and CTP (PerkinElmer Life Sciences, Boston, Mass.). aRNA was quantified by absorbance at 260 nm.

Gene specific sense oligonucleotides (25-mers) were synthesized with 5′-amino uni-linkers and coupled to Luminex xMAP carboxylated microspheres according to the manufacturer's protocol (Luminex Corp., Austin, Tex.). Each gene specific oligonucleotide was coupled to a distinct colored/numbered microsphere; 1 nmole of oligonucleotide was coupled to 2.5×10⁶ microspheres in a single reaction and suspended in 100 μl of 10 nM Tris/0.1 mM EDTA, pH 8.0. The microspheres were tittered using a hemacytometer.

For hybridization of aRNA to capture probe-coupled microspheres, 5,000 microspheres of each gene were pooled, mixed, and suspended in 60 μl of hybridization buffer with 10 μg of aRNA that had been previously randomly fragmented by heating at 94° C. for 35 min. The samples were hybridized at 60° C. for 4-5 hours with constant mixing. Hybridizations were performed in 3M tetramethylammonium chloride (TMAC) (Sigma, St. Louis, Mo.), 50 mM Tris pH 8.0, and 4 mM EDTA, pH 8.0. Following washing on a vacuum manifold to remove unbound aRNA, mixtures were incubated with streptavidin-R-phycoerythrin conjugate for 15 min at room temperature with shaking at 400 RPM, washed, and resuspended in 75 μl of wash buffer (1×PBS, 1 mM EDTA, 0.01% Tween 20).

The microspheres were analyzed on a Luminex 100 xMAP system (Luminex Corp., Austin, Tex.) and at least 200 events of each set of individually colored microspheres were counted.

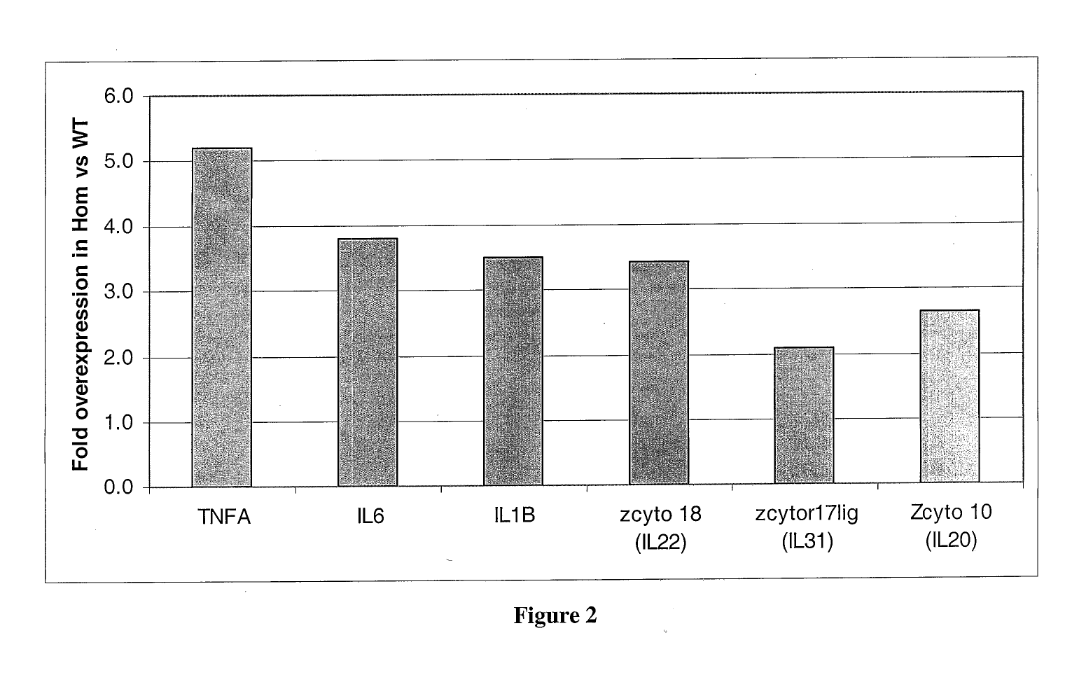

Many genes failed to show any robust differential expression between wild type and knockout mice during the course of the study. However, the knockout animals did exhibit up-regulation of transcripts for a number of cytokine and chemokine genes in tissues. Of 42 cytokine or chemokine transcripts profiled at day 7 post wounding, 36% showed greater than two fold up-regulation in the knockout when compared to the wild-type. These included TNF-α, IL-6, IL-1β, IL-20 (zcyto10), IL-22 (zcyto18), and IL-31 (zcytor17lig). A sample data set with up-regulation of these genes at day seven post wounding is shown in FIG. 2.

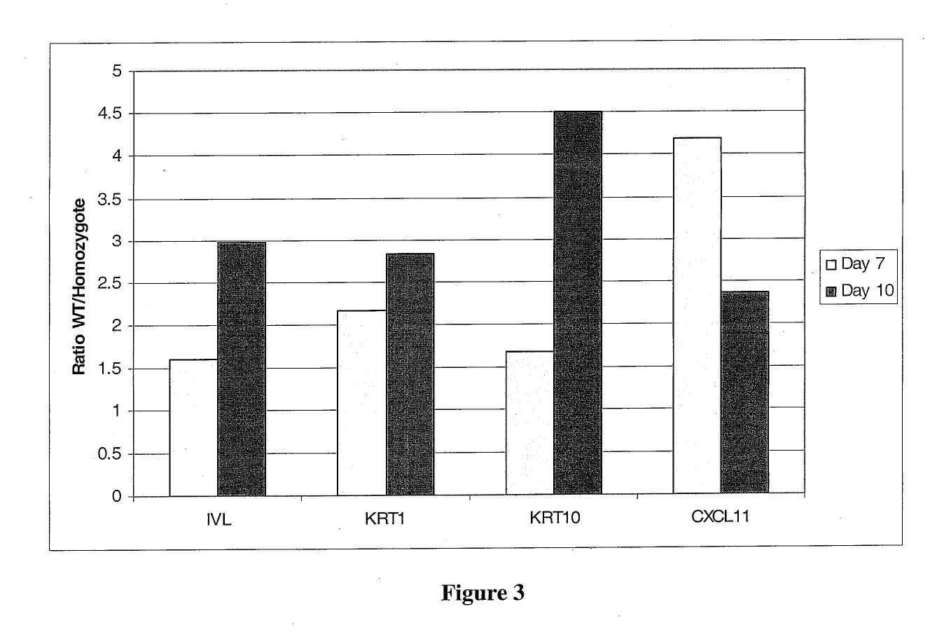

In contrast to the overexpression of inflammatory cytokines in knockout tissue, there was an under-representation of transcripts associated with fully differentiated epidermis, suggesting that the formation of a fully differentiated epidermis was retarded in the IL-17B (zcyto7) knockout environment. In particular keratin 1 (KRT1), keratin 10 (KRT10), and involucrin (IVL), all of which are associated with differentiated epidermis, were under-represented in knockout when compared to wild-type animals. In addition, there was also decreased expression of CXCL11, a chemokine previously reported to be required for mobilization of keratinocyes and their migration in a wound environment. A sample data set with down-regulation of transcripts associated with fully differentiated epidermis is shown in FIG. 3.

### EXAMPLE 3

The mouse model of cutaneous leishmaniasis was performed essentially as described in “Animal models for the analysis of immune responses to leishmaniasis,” in Current Protocols in Immunology, David Sacks and Peter Melby, Chapter 19.2.1-19.2.20 (1998). This model was used to investigate the role of zcyto7 in wound healing.

Historically, susceptibility to cutaneous L. major infection has been associated with chronic and progressive swelling at the site of infection, development of Th2 responses (low IFN-g:IL-4 production ratio; high levels of IL-4 produced) and production of high levels of IL-10, elevated levels of serum IgE and systemic dissemination of L. major. Resistance to cutaneous L. major infection has been associated with acute swelling at the site of infection that ultimately heals, development of Th1 responses (high IFN-g:IL-4 production ratio), absence of serum IgE and containment of L. major to the site of infection.

Recent publications have shown that CD4⁺ T cell responses (Th1 vs. Th2) to L. major are not the only factor that determines resistance vs. susceptibility in the mouse model of cutaneous L. major infection. For example, genetic defects in wound-healing have recently been suggested to explain why some strains of mice are resistant to L. major, including development of Th1 responses, but develop more severe and prolonged swelling at the site of infection (Sakthianandeswaren et al., (2005) PNAS 102 (43): 15551-15556). Alternatively, defects in neutrophil recruitment to the site of infection may result in a similar L. major disease phenotype in C57B1/6 mice (Ribeiro-Gomes et al., (2004) J. Immunol. 172: 4454-4462).

All mice were female and age-matched. The C57B1/6-congenic homozygous zcyto7 wild-type and zcyto7 gene-targeted (“zcyto7 knockout”) mice were obtained from in-house stocks. The zcyto7 congenic lines had been derived by in-house backcrossing of heterozygous zcyto7 knockout mice (OzGene, Bentley, Australia) to C57B1/6 mice. C57B1/6 and BALB/c control mice were purchased from Charles River Laboratories, Wilmington, Mass.

Leishmania major (L. major, strain WHOM/IR/-/173) was cultured in vitro from frozen stocks. Infectious L. major promastigotes were prepared by PNA-selection performed by incubation of cultured promastigotes (4×10⁸/ml) with PNA-coated agarose beads (1:20 dilution; Sigma, St. Louis, Mo.) followed by differential sedimentation to pellet PNA-bound promastigotes. Free promastigotes in the supernatant were collected, washed, counted and resuspended in PBS at the appropriate concentration for infection of mice.

Mice (n=5/group) were injected subcutaneously in one hind footpad with 1×10⁶ infectious L. major promastigotes in 30 ul PBS on day 0 of the model. Disease progression was followed weekly for 12 weeks by measuring footpad thickness with a metric caliper, measuring body weights with a lab scale and clinical scoring of footpad lesions by eye. Clinical scoring: 0=no lesion, 1=open lesion of <1 mm, 2=open/necrotic lesion covering part of footpad (˜1-4 mm), 3=open/necrotic lesion covering majority of footpad (>4 mm). Serum was collected by eye-bleed at day −2, week 6 and week 12 of the model. At designated time-points, mice were killed and serum, spleens and draining popliteal lymph-nodes were collected for in vitro analysis. The BALB/c mice were killed and serum collected at week 6 post-infection due to the severity of their L. major disease at this time point. Spleens and lymph-nodes were not collected for in vitro analysis from BALB/c mice.

L. major lysate antigen was prepared by repeated freeze-thaw of a sterile, high-density suspension of L. major promastigotes in PBS followed by high-speed centrifugation to remove debris. Lysate supernatants were stored in single-use aliquots at −80° C. Lack of residual viable L. major was verified by microscopic inspection and by in vitro culture. Protein concentration was estimated using a BCA kit (Pierce). Optimal dilutions of lysate for T cell stimulation in vitro were identified in preliminary [3H]-incorporation experiments.

Single-cell suspensions of spleen and lymph-node lymphocytes were prepared in culture medium (RPMI+10% FCS). Spleen and lymph-node cells (5×10⁵/well) from each group of mice were pooled and cultured at 37° C. in flat-bottom 96-well plates in triplicate wells with either medium, L. major lysate antigen (1:100 and 1:200 dilutions) or ConA (0.5 ug/ml). Cell supernatants were collected at 48 hours for analysis of cytokine levels using a Luminex kit according to the manufacturer's instructions. Cells were pulsed with 1 uCi/well of [3H]-thymidine for an additional 12 hours, and then harvested for analysis of CPM of [3H]-incorporated using a TopCount beta counter. Data are plotted as the mean CPM for each antigen for each group of mice.

Relative levels of L. major-specific serum IgG1 and IgG2a were quantitated by ELISA. ELISA plates were coated overnight with L. major antigen (3.4 ug/ml) in PBS. The plates were blocked with PBS+1% BSA, washed, and then incubated for 2-3 hours with serum samples serially diluted in PBS+1% BSA. The plates were developed by serial 1 hour incubations with biotinylated goat anti-mouse IgG1 or IgG2a antibody (Southern Biotech, Brmingham, Ala.), streptavidin-horseradish peroxidase conjugate (Jackson Immunoresearch, West Grove, Pa.) and HRP substrate (TMB One Solution; Promega, Madison, Wis.). Color development was halted by addition of 0.1 N HCl. The absorbance of each well was read at both 450 & 630 nanometers using a Spectra MAX 190 ELISA plate reader (Molecular Devices, Sunnyvale, Calif.). Data are plotted as [A₄₅₀-A₆₃₀] on the Y axis versus 1/dilution of serum on the X axis.

Relative levels of total serum IgE were quantitated by ELISA. ELISA plates were coated overnight with IgE-specific goat anti-mouse IgE antibody (Southern Biotech, Birmingham, Ala.). The plates were blocked with PBS+1% BSA, washed, and then incubated for 2-3 hours with serum samples serially diluted in PBS+1% BSA. The plates were developed by serial 1-hour incubations with biotinylated goat anti-mouse IgE antibody (Southern Biotech, Birmingham, Ala.), streptavidin-horseradish peroxidase conjugate (Jackson Immunoresearch, West Grove, Pa.) and HRP substrate (TMB One Solution; Promega, Madison, Wis.). Color development was halted by addition of 0.1 N HCl. The absorbance of each well was read at both 450 & 630 nanometers using a Spectra MAX 190 ELISA plate reader (Molecular Devices, Sunnyvale, Calif.). Data are plotted as [A₄₅₀-A₆₃₀] on the Y axis versus 1/dilution of serum on the X axis.

Control BALB/c mice were susceptible to L. major and developed severe/progressive L. major disease as would be expected for this strain. Signs of progressive disease included progressive swelling of the infected footpads that did not resolve, the development of large open lesions on the infected footpads and the failure to gain weight over time. These mice also had high levels of total IgE in their serum.

Control C57B1/6 mice were resistant to L. major, developed limited footpad swelling that resolved by 8 weeks post-infection, and gained body weight normally as would be expected for this strain. They also developed Th1 responses, characterized by a high ratio of IFN-gamma:IL-4 production to L. major antigen in vitro and a high ratio of IgG2a:IgG1 L. major-specific antibody and an absence of IgE in their serum.

C57B1/6-congenic zcyto7 wild-type mice had an L. major disease phenotype that was indistinguishable from that of C57B1/6 control mice. They were resistant to L. major and developed moderate footpad swelling that resolved by 8 weeks post-infection. They also developed Th1 responses, characterized by a high ratio of IFN-gamma:IL-4 production to L. major antigen in vitro and a high ratio of IgG2a:IgG1 L. major-specific antibody and an absence of IgE in their serum.

The C57B1/6-congenic zcyto7 gene-targeted mice were resistant to L. major and gained body weight normally. However they developed significantly larger footpads that took significantly longer (>12 weeks) to resolve than did footpads in C57B1/6 and zcyto7 wild-type mice. Development of small open lesions also developed on their footpads; no lesions were observed on the footpads of C57B1/6 and zcyto7 wild-type mice. They had larger spleens and draining lymph-nodes at 12-weeks, which is consistent with their having more severe symptoms of disease than the control mice at this time-point. They developed Th1 responses, characterized by a high ratio of IFN-gamma:IL-4 production to L. major antigen in vitro and a high ratio of IgG2a:IgG1 L. major-specific antibody and an absence of IgE in their serum antibody.

This data suggest that zcyto7 is not required for development of Th1 responses to L. major, but rather may be important for wound-healing or immune control of L. major infection in vivo.

### EXAMPLE 4

### IL17B Knockout Mice Exhibit Altered Disease Progression in a DSS Colitis Model

To investigate disease susceptibility mice were run through the dextran sulfate sodium (DSS) model of colitis. This model induces an acute colitis which is manifest by bloody diarrhea, weight loss, shortening of the colon and mucosal ulceration with neutrophil infiltration. DSS-induced colitis is characterized histologically by infiltration of inflammatory cells into the lamina propria, with lymphoid hyperplasia, focal crypt damage, and epithelial ulceration. These changes are thought to develop due to a toxic effect of DSS on the epithelium and by phagocytosis of lamina propria cells and production of TNF-alpha and IFN-gamma.

To induce DSS colitis mice were treated with a 2-2.5% solution of reagent grade dextran sulfate sodium (DSS, MP Biochemicals, Solon, Ohio), molecular weight 36,000-50,000 administered ad libitum in drinking water. Animals received this DSS drinking water for 5 days and were then returned to normal water. Using this model both onset of colitis in response to DSS treatment and subsequent recovery after DSS withdrawal can be measured. Disease progression can be monitored during the course of the study by loss of weight. In a typical study normal mice will lose 5-10% of bodyweight within 7-8 days of initiating DSS treatment but will return to a normal weight after 5 days on non-DSS drinking water. As indicated in Table 1, in this model IL17B knockout mice exhibited an increased weight loss at the peak of disease. In addition IL17B knockout mice exhibited a retarded recovery upon transfer to normal water: after 5 days on normal water wild type animals but not IL17B knockout mice had regained weight lost during the course of the study.

TABLE 1

| | Peak weight loss (% body | Weight change upon recovery (5 |
|---|---|---|
| Genotype | weight) | days normal water) |
| Wild type | 7.6% | 0.3% |
| Knockout | 14.2% | 5.8% |

Thus the lack of IL17B results in exacerbated disease in the DSS colitis model. Such a phenotype could be caused by the failure of immune cells or epithelial cells to modulate or repair the damage and inflammation inherent in this model.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

We claim:

1. A method for promoting wound healing in a patient in need of such treatment comprising administration of IL-17B.

2. The method of claim 1 wherein the IL-17B is the product of eukaryotic host cell expression.

3. The method of claim 1 wherein the IL-17B is the product of prokaryotic host cell expression.

4. The method of claim 3 wherein the prokaryotic host cell is E. coli.

5. The method of claim 1 wherein the wound type is selected from the group consisting of mechanical, thermal, acute, chronic, infected, and sterile wounds.

6. The method of claim 1 wherein the wound healing is for treatment of a disease selected from the group consisting of mucositis, cutaneous pressure ulcers, diabetic skin wounds, and inflammatory bowel disease.

7. The method of claim 1 wherein the patient is human.

8. The method of claim 1 further comprising administering at least one other factor selected from the group consisting of GM-CSF, CSF, EGF, a FGF, KGF, PD-ECGF, PDGF, TGF-α, TGF-β, an IL, an IFN, IGF-I, IGF-II, KGF, M-CSF and SCF.

9. The method of claim 1 wherein the administration is selected from the group consisting of topical, subcutaneous, intravenous, intramuscular, or intraperitoneal.

10. The method of claim 8 wherein the administration is topical.

11. The method of claim 9 wherein the topical administration of IL-17B is conducted through the application of an IL-17B comprising wound covering selected from the group consisting of a collagen based cream, a collagen based film, a collagen based microcapsule, a collagen based powder, hyaluronic acid or other glycosaminoglycans, creams, foams, suture material, and wound dressing.

12. The method of claim 10 further comprising administering at least one other factor selected from the group consisting of GM-CSF, CSF, EGF, a FGF, KGF, PD-ECGF, PDGF, TGF-α, TGF-β, an IL, an IFN, IGF-I, IGF-II, KGF, M-CSF and SCF.

13. The method of claim 9 wherein topical administration of IL-17B is conducted through the application of a solution comprising IL-17B.

14. The method of claim 12 further comprising administering at least one other factor selected from the group consisting of GM-CSF, CSF, EGF, a FGF, KGF, PD-ECGF, PDGF, TGF-α, TGF-β, an IL, an IFN, IGF-I, IGF-II, KGF, M-CSF and SCF.

15. A pharmacological composition comprising IL-17B in combination with one or more pharmaceutically acceptable carriers or adjuvants.

16. The composition of claim 14 further comprising at least one other factor selected from the group consisting of GM-CSF, CSF, EGF, a FGF, KGF, PD-ECGF, PDGF, TGF-α, TGF-β, an IL, an IFN, IGF-I, IGF-II, KGF, M-CSF and SCF.

17. A method for the treatment of a disease involving dyregulation of epithelial repair comprising the administration of an antagonist of IL-17B.

18. The method of claim 17 wherein said disease is psoriasis.

19. The method of claim 17 wherein said antagonist is an antibody that specifically binds to IL-17B.
